(19) United States
(12) Patent Application Publication
(10) Pub. No.: US 2015/0127017 A1
(43) Pub. Date: May 7, 2015

(54) ELECTRICAL STIMULATION OF THE CERVIX

(71) Applicants: Robert E. Garfield, Goodyear, AZ (US); Shao-Qing Shi, Goodyear, AZ (US); Leili Shi, Goodyear, AZ (US); William Maner, LaMarque, TX (US)

(72) Inventors: Robert E. Garfield, Goodyear, AZ (US); Shao-Qing Shi, Goodyear, AZ (US); Leili Shi, Goodyear, AZ (US); William Maner, LaMarque, TX (US)

(21) Appl. No.: 14/401,042

(22) PCT Filed: May 20, 2013
(86) PCT No.: PCT/US2013/041855

§ 371 Date: Nov. 13, 2014

Related U.S. Application Data
(60) Provisional application No. 61/649,128, filed on May 18, 2012.

(51) Int. Cl. A61N 1/36; A61B 17/42

(52) U.S. Cl. CPC A61N 1/36007; A61B 17/42
USPC 606/121

(57) ABSTRACT

Systems and methods for producing cervical ripening in a pregnant patient. The method includes generating an electrical stimulation current between about 0.01 milliamperes and about 6 milliamperes using a current unit, coupling an electrode probe to the current unit, and inserting the electrode probe transvaginally so that at least one electrode of the electrode probe is in contact with the patient's cervix. The method also includes applying the electrical stimulation current from the current unit through the electrode probe and the at least one electrode to the patient's cervix to produce ripening of the patient's cervix.

### CROSS REFERENCE TO RELATED APPLICATIONS

The application is based on, claims priority to, and incorporates herein by reference U.S. Provisional Application Ser. No. 61/649,128, filed May 18, 2012, and entitled, “Electrical Stimulation of the Cervix.”

### STATEMENT REGARDING FEDERALLY SPONSORED RESEARCH

N/A.

### BACKGROUND OF THE INVENTION

The present application is directed toward systems and methods for electrical stimulation of a cervix. In particular, the present application relates to electrical stimulation for softening or ripening a cervix during pregnancy.

In preparation for labor and delivery, chemical and physical changes cause the cervix to soften through a process called cervical ripening. Specifically, cervical ripening encompasses three stages of cervical changes including softening (as shown in FIG. 1A), dilation (as shown in FIG. 1B), and effacement (as shown in FIG. 1C). Cervical ripening is important for successful vaginal birth because this process prepares the cervix for passage of the fetus.

Successful vaginal birth is less likely to occur in the absence of a ripe or favorable cervix and, as a result, cervical ripening treatments are commonly applied to facilitate labor induction. The type of treatment to be applied is often determined after assessment of the cervix (for example, by calculating a Bishop score). Nonpharmacologic treatments include herbal compounds, castor oil, hot baths, enemas, sexual intercourse, breast stimulation, acupuncture, acupressure, transcutaneous nerve stimulation, and mechanical and surgical modalities. Of these nonpharmacologic methods, only the mechanical and surgical treatments have proven efficacy for cervical ripening or induction of labor. However, these surgical and mechanical methods have associated risks such as infection, bleeding, membrane rupture, and placental disruption. Pharmacologic agents available for cervical ripening and labor induction include oxytocin, prostaglandins, misoprostol, mifepristone, and relaxin. The application of pharmacological agents, however, is not well-controlled and can have dangerous side effects for the mother.

Therefore, there is a need for a safe and effective method for cervical ripening and labor induction.

### SUMMARY OF THE INVENTION

The present invention provides a system for producing cervical ripening in a pregnant patient. The system includes a stimulation device, a current unit, and an electrode probe. The stimulation device is configured to perform preprogrammed stimulation tasks or user-defined stimulation tasks to generate stimulation current parameters. The current unit is configured to be controlled by the stimulation device to produce a stimulating current based on the stimulation current parameters. The electrode probe is coupled to and receives stimulation current from the current unit. The electrode probe provides the stimulating current through at least one stimulation electrode to the cervix of the patient to cause ripening of the cervix.

A method according to the present invention for producing cervical ripening in a pregnant patient includes generating an electrical stimulation current between about 0.01 milliamperes and about 6 milliamperes using a current unit, coupling an electrode probe to the current unit, and inserting the electrode probe transvaginally so that at least one electrode of the electrode probe is in contact with the patient's cervix. The method also includes applying the electrical stimulation current from the current unit through the electrode probe and the at least one electrode to the patient's cervix to produce ripening of the patient's cervix.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIGS. 1A-1C illustrate a softening stage, a effacement stage, and a dilation stage, respectively, of cervical ripening;

FIG. 2 is a block diagram of a system, according to the present invention, for applying electrical stimulation to a cervix to produce cervical ripening;

FIGS. 3A and 3B are perspective views of electrode probes for use with the system of FIG. 2;

FIG. 4 is a graph illustrating collagen light-induced fluorescence over a period of gestation days in control rats and rats that received treatment according to methods of the present invention; and

FIG. 5 is a graph illustrating cervical length after application gram weights to control rats and rats that received treatment according to methods of the present invention.

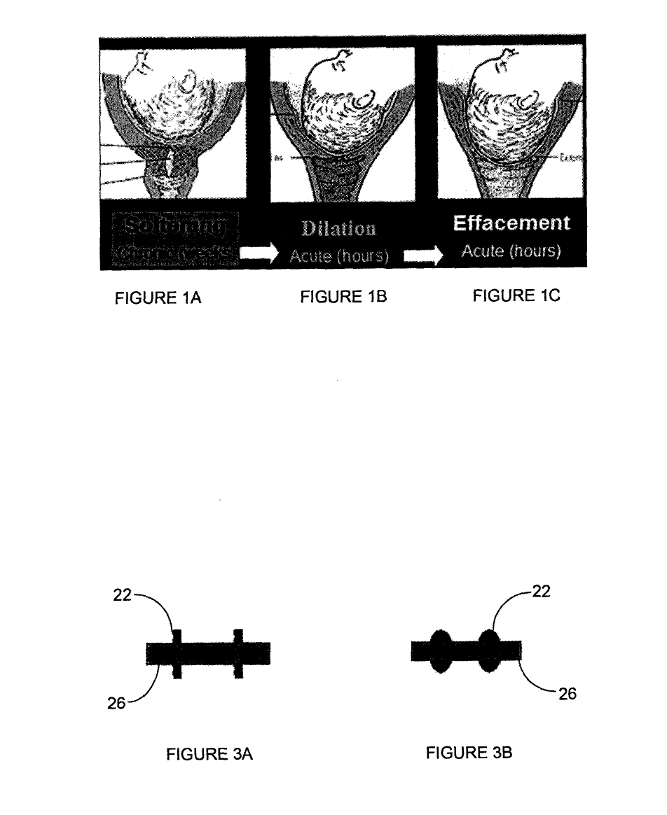

### DETAILED DESCRIPTION OF THE INVENTION

According to systems and methods of the present invention, electrical stimulation of the cervix can produce cervical ripening to facilitate labor and delivery of a pregnant patient (for example, at the end of gestation). More specifically, systems and methods of the present invention include the application of electrical stimulation current to the cervix, resulting in softening and ripening of the cervix to help vaginal delivery of a fetus. The electrical stimulation current may be provided using stimulation parameters below perception levels in order to ripen the cervix without pain or damage to the patient.

Electrical stimulation can be used to activate neural and/or inflammatory pathways or other systems of the cervix in order to induce the release of prostaglandins or other mediators, resulting in softening and ripening of the cervix. This offers a more localized and accurate method of treatment in comparison to conventional mechanical methods, which can be inconsistent and can also harm the placenta and other nearby membranes, as well as pharmacological methods, which cannot be easily terminated and generally affect the whole body rather than just the cervix. Some methods of the present invention include using electrical stimulation as a primary ripening treatment alone or in combination with conventional mechanical treatments or pharmacologic or uterotonic agents.

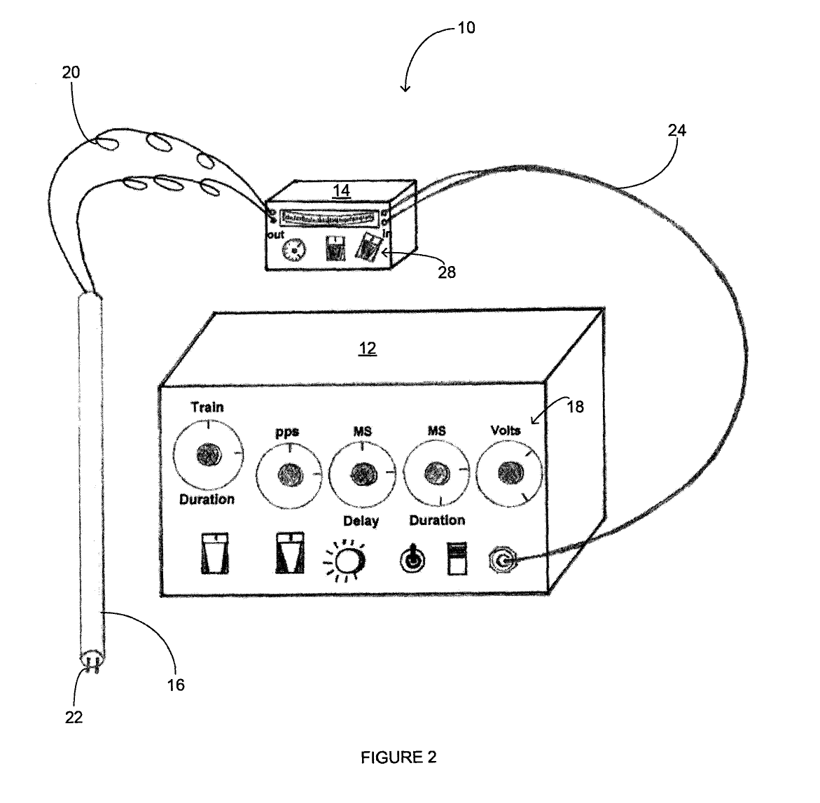

An example system 10 for carrying out such methods of electrical stimulation for cervical ripening is illustrated in FIG. 2. The system 10 can include a stimulation device 12, a constant current unit 14, and an electrode probe 16. The stimulation device 12 can provide user controls 18 for setting electrical stimulation parameters of a stimulation current, such as frequency, amplitude, pulse width, and train duration for selected periods of time as well as operating voltage and/or other parameters. The stimulation device 12 can communicate with the constant current unit 14 (such as through wired or wireless connections 24) so that the constant current unit 14 generates and provides the stimulation current to the electrode probe 16 via lead wires 20 based on the electrical stimulation parameters.

The electrode probe 16 can be inserted transvaginally until electrodes 22 come in contact with a patient's cervix. In some implementations of the system 10, the electrode probe 16 can include bar electrodes 22, rod electrodes 22 (as shown in the bipolar electrode disk 26 of FIG. 3A, which may be connected to an end of the electrode probe 16), or needle electrodes 22. In one implementation, the electrode probe 16 can include a bipolar electrode disk 26 (such as a button electrode) with rounded end, or bead electrodes 22, as shown in FIG. 3B, in order to prevent damage or tearing of the cervical tissue. In addition, the electrodes 22 can be constructed of copper or, alternatively, stainless steel or platinum-iridium metals to prevent potential copper electro-deposition from damaging the cervix. Also, in one implementation of the system 10, the lead wires 20 can be manufactured by Advantage Medical Cables.

The stimulation device 12 can contain computing capability, software, and memory or storage. The stimulation device 12 can be set using the user controls 18, such as dials, switches and/or auxiliary inputs, to perform preprogrammed stimulation tasks, each including commanding the constant current unit 14 to generate and output stimulation current of selected frequency, amplitude, pulse width, and train duration automatically for selected periods of time. The stimulation device 12 can also be operated manually by a user, thereby performing user-defined stimulation tasks, in which the user can determine and set one or more output stimulation currents of desired frequencies, amplitudes, pulse widths, and train durations as needed spontaneously (that is, in real time or in near-real time). For example, the stimulation device 12 can be operated automatically or manually to control a stimulation current that can cause cervical ripening, and the user has the capability to adjust the stimulation current parameters (such as frequencies, amplitudes, pulse widths, train durations, and/or accompanying voltages) in real time or near-real time during observation of the patient's cervix.

During some operations, the stimulation device 12 can automatically or manually operate multiple stimulation outputs of the constant current unit 14 independently or in unison with varying or similar current frequencies, amplitudes, pulse widths, and train durations. As a result, the system 10 can provide stimulation currents directly to the cervix and through various other organs, such as the vaginal wall and/or abdominal wall separately, simultaneously, or sequentially, or can provide stimulation currents to various parts of the cervix separately, simultaneously, or sequentially.

In some implementations, pre-recorded electrical traces, obtained from normally ripening patients and saved digitally, can be stored in the stimulation device 12 to be used as the electrical current trace patterns (that is, the stimulation current parameters) for commanding the constant current unit 14 to output identical stimulation current to patients with insufficient cervical ripening. In addition, artificially generated current traces, saved digitally, with known frequencies, amplitudes, pulse widths, and train durations, can be stored in the stimulation device 12 to be used as the electrical current trace patterns for commanding the constant current unit 14 to output identical stimulation current to patients with insufficient cervical ripening. These pre-recorded electrical traces and artificially generated current traces may be considered preprogrammed stimulation tasks.

In another implementation, the stimulation device 12 can automatically regulate and modify the output produced by the constant current unit 14 based on input from electrical activity of the patient's cervix, which can be transmitted to the stimulation device 12 via pick-up wires, a signal conditioner, and/or after-conditioning wires (not shown). The stimulation device 12 can regulate and modify the produced electrical current by changing the electrical stimulation pulse-width, current amplitude, pulse train duration, and/or the pulse frequency according to a pre-programmed algorithm.

In some implementations, the stimulation device 12 can include a display (not shown), such as a video display, a digital display, light-emitting diode (LED) display, and the like, to display the stimulation currents produced for the user to read or assess. The stimulation device 12 can be coupled to the constant current unit 14 by wires, direct electrical coupling, a wireless connection (such as Bluetooth®), or another suitable coupling.

The constant current unit 14 can generate the output stimulation current. The electrical stimulation current settings can be adjusted manually at the constant current unit 14 by the user using user controls 28, such as dials, switches or other devices, as shown in FIG. 2. In addition, or alternatively, the electrical stimulation settings can be controlled by the stimulation device 12 (for example, as preprogrammed settings or by the user using the user controls 18, as described above) and output to the constant current unit 14. As described above, in some operations, the constant current unit 14 can output multiple electrical stimulation currents directly to the cervix and/or to other organs such as the vaginal wall and/or the abdominal wall separately, simultaneously, or sequentially, as commanded by the stimulation device 12, or the constant current unit 14 can independently output multiple electrical stimulation currents to various locations of the cervix separately, simultaneously, or sequentially.

In some implementations, there can be a constant two-way communication between the constant current unit 14 and stimulation device 12, so that the constant current unit 14 can receive commands from the stimulation device 12 and the stimulation device 12 can receive actual output simulation current values from the constant current unit 14.

Example operating parameters generated and output by the constant current unit 14 include an output current about 0.01 milliamperes and about 100.00 milliamperes (with possible voltages between about 0.0001 volts and about 100 volts). Pulse widths of the current can be adjusted between about 0.1 millisecond and about 5000 milliseconds. Frequencies of the current can be adjusted from about 0.1 Hertz to about 30 Hz or greater, or about 100 Hz or greater. Pulse train durations can be adjusted from about 1 second to about 60,000 seconds. In addition, output currents can be sinusoidal so as to reduce potential tissue damage. The constant current unit 14 may also be capable of generating a maximal “jolt” of uterine electrical stimulation energy equivalent to between about 1 Joule and about 120 Joules of electrical energy in a short duration between about 1 millisecond and about 1000 milliseconds. Further, the electrical stimulation current output from the constant current unit 14 can be sensed, measured, or detected by either the constant current unit 14 or the stimulation device 12 and can be automatically shut off if current values are determined to be dangerous or outside prescribed, programmed, or set values.

In some implementations, parameters can be set for inducing cervical ripening so that the electrical stimulation is well below perception levels of the patient (such as by using current within a range between about 0.01 milliamperes and about 6 milliamperes or voltage within a range between about 0.1 volts and about 60 volts). For example, electrical stimulation can be applied to the cervix at levels below perception for about 1 minute, about 1 hour, about 5 or 6 hours, or any time in between, to facilitate cervical ripening and/or labor induction. Example parameters in animal studies, as further described below, include voltages of about 1, 5, 10, or 20 volts, about 5000 millisecond durations, about 0.01 millisecond delays, about 0.1, 0.5, 1.0, 2.0, or 5.0 hertz or pulses per second (pps), about 10,000 or about 60,000 millisecond train durations (with or without alternating on/off periods), and about 0.1 milliamperes current to about 10.0 milliamperes current.

The ability to control and monitor output stimulation currents, as described above, allows for an accurate application of cervical ripening treatment. For example, electrical stimulation can be quickly terminated through control of the constant current unit 14 or the stimulation device 12, whereas conventional pharmacological or uterotonic agents cannot be accurately controlled or their side effects immediately terminated. Additionally, since stimulation current is applied directly to the cervix via the electrode probe 16, only the cervix is affected by this form of treatment, in comparison to manual stimulation, which may affect the placenta or adjacent tissues, or pharmacological agents that affect the entire body.

In some implementations of the present system 10, the stimulation device 12 and the constant current unit 14 can be a single, integral unit or can be housed together an integral unit. In addition, in some implementations, the stimulation device 12 and/or the constant current unit 14 can include additional integral components, such as an isolation component, a constant maximum current component, and/or a biphasic converter component. For example, the isolation component can prevent ground loop currents from affecting the patient. This may be accomplished through optical isolation, induction, or other suitable isolation methods.

The constant maximum current component can allow the user to regulate the amount of maximum current that the patient's cervix receives. This can prevent tissue damage due to extreme current fluctuations as tissue resistance varies, and can be set (either in a discrete or continuous fashion) to or between values well below human threshold for human feeling (for example, about 0.01 milliamperes to about 6 milliamperes) and values uncomfortable for humans (for example, about 100 milliamperes). In one example, the constant maximum stimulation current can be set at a value which maximizes current input without damaging tissue and with minimal discomfort to the patient (such as about 4 milliamperes).

The biphasic converter component can alternate the polarity of current pulses produced by the constant current unit 14 after having moved through the isolation component and the constant maximum current component in order to further prevent adverse effects on the patient's tissues. The biphasic converter component can ensure that the total energy delivered at the tissue site, as integrated over time, has a net value of zero. This can reduce the possibility of heating and subsequent damage to the patient's tissues.

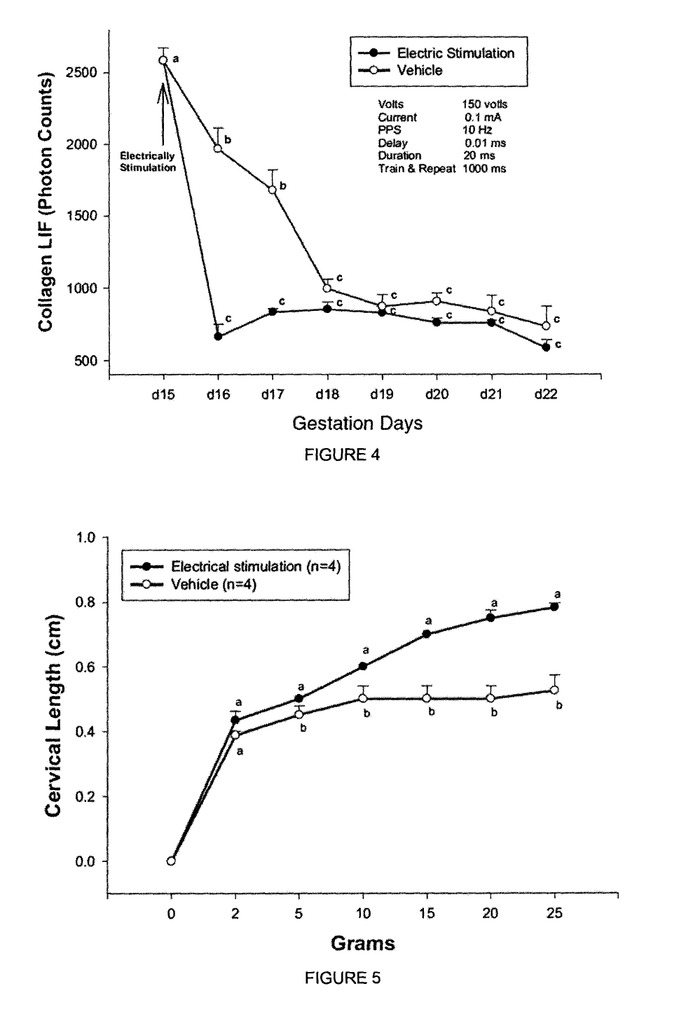

FIGS. 4 and 5 illustrate results of testing methods of the present invention using rats. In the tests, current pulses of 100 microamperes, 10 pulses per sec, and 20 milliseconds per pulse were applied through electrodes to rat cervices for about 2 hours on day 15 of gestation. Control rats received an electrode probe placed on their cervix without any current applied. Daily measurements of cervical light-induced fluorescent (LIF, photon counts of collagen x-bridge fluorescence) via a collascope were made on days 16 of gestation until spontaneous delivery (day 22) to estimate changes in cervical ripening. As shown in FIG. 4, both sets of animals (that is, treated rats and control rats) showed significant decline of LIF values from day 15 of gestation and reached the lowest levels during delivery. However, LIF values were significantly lower in treated animals on days 16 and 17 of gestation as compared to control animals. FIG. 5 shows that the cervix of treated rats was less resistant to stretch, or more extensible, (that is, due to greater cervical ripening) at each applied gram weight as compared to the control. In other words, FIG. 5 illustrates that cervical extensibility, as well as cervical length after application of gram weights, was significantly greater in treated animals as compared to control animals. The results of the testing described above illustrate that low levels of electrical stimulation (that is, well below perception and not causing pain or damage) ripen the cervix. Further, following electrical stimulation, the cervix remains ripened until delivery. The results also illustrated that electrical stimulation did not produce early birth or affect the fetus weight, nor did electrical stimulation cause visual changes or damage to the cervix.

The present invention has been described in terms of one or more preferred embodiments, and it should be appreciated that many equivalents, alternatives, variations, and modifications, aside from those expressly stated, are possible and within the scope of the invention.

1. A system for producing cervical ripening in a pregnant patient, the system comprising:
a stimulation device configured to perform at least one of preprogrammed stimulation tasks and user-defined stimulation tasks to generate stimulation current parameters;
a current unit configured to be controlled by the stimulation device to produce a stimulation current based on the stimulation current parameters;
an electrode probe coupled to the current unit and configured to receive the stimulation current, the electrode probe including at least one stimulation electrode configured to be coupled to the cervix of the patient, the electrode probe providing the stimulation current through the at least one stimulation electrode to the cervix to cause ripening of the cervix.

2. The system of claim 1, wherein one of the stimulation device and the current unit includes an isolation component configured to prevent ground loop currents within the stimulating current produced by the current unit from adversely affecting the patient.

3. The system of claim 1, wherein one of the stimulation device and the current unit includes a maximum current component configured to regulate an amount of the stimulating current produced by the current unit to be applied to the patient's cervix in order to prevent tissue damage to the patient.

4. The system of claim 1, wherein one of the stimulation device and the current unit includes a biphasic converter component configured to alternate a polarity of the stimulating current produced by current unit so that the stimulating current, as integrated over time, has a net value of zero in order to prevent tissue damage to the patient.

5. The system of claim 1, wherein the current unit is configured to produce the stimulation current with at least one of a current amplitude ranging from about 0.01 milliamperes to about 40.00 milliamperes, with a voltage ranging from about 0.1 volts to about 100 volts, with pulse widths ranging from about 0.1 milliseconds to about 5000 milliseconds, with frequencies from about 0.1 Hertz to about 30 Hertz, and with pulse train durations ranging from about 1 second to about 60,000 seconds.

6. The system of claim 5, wherein the current unit is configured to produce the stimulation current with at least one of a current amplitude ranging from about 0.01 milliamperes to about 6 milliamperes, with a voltage ranging from about 0.0001 volts to about 60 volts, and with frequencies from about 0.1 Hertz to about 5.0 Hertz.

7. The system of claim 1, wherein the stimulation device is configured to monitor the stimulation current from the current unit and automatically shut down the current unit if parameters of the stimulation current are outside of one of prescribed, programmed, and set values.

8. The system of claim 1, wherein the stimulation device is configured to control the current unit through one of wired connections and wireless connections.

9. The system of claim 1, wherein the stimulation device includes user controls configured to receive user input, wherein the stimulation device is configured to generate the stimulation current parameters based on the user input.

10. The system of claim 1, wherein the current unit includes user controls configured to receive user input, wherein the current unit is configured to modify the stimulation current based on the user input.

11. The system of claim 1, wherein the electrode probe is coupled to the current unit through lead wires.

12. The system of claim 1, wherein the at least one stimulation electrode includes a bipolar electrode disk including one of rod electrodes and bead electrodes.

13. The system of claim 1, wherein the preprogrammed stimulation tasks include one of pre-recorded electrical traces obtained from normally ripening patients and artificially generated current traces.

14. The system of claim 1, wherein the stimulation device is configured to receive electrical activity of the patient's cervix and to modify the stimulation current parameters based on the electrical activity.

15. The system of claim 1, wherein the stimulation device includes a display and is configured to display at least the stimulation current parameters through the display.

16. A method for producing cervical ripening in a pregnant patient, the method comprising:
generating an electrical stimulation current between about 0.01 milliamperes and about 6 milliamperes using a current unit;
coupling an electrode probe to the current unit;
inserting the electrode probe transvaginally;
contacting at least one electrode of the electrode probe against the patient's cervix; and
applying the electrical stimulation current from the current unit through the electrode probe and the at least one electrode to the patient's cervix to produce ripening of the patient's cervix.

17. The method of claim 16 and further comprising administering a uterotonic agent to the patient.

18. The method of claim 16, wherein the step of applying the electrical stimulation current further includes applying the electrical stimulation current for a duration between about 1 minute to about 6 hours.

19. The method of claim 16 and comprising receiving input from electrical activity of the patient's cervix in response to applying the electrical stimulation current, and modifying the electrical stimulation current based on the electrical activity.

20. The method of claim 16 and further comprising obtaining pre-recorded electrical traces from a normally ripening cervix and generating the electrical stimulation current based on the pre-recorded electrical traces.
